(19) United States
(12) Patent Application Publication
(10) Pub. No.: US 2012/0107854 A1
(43) Pub. Date: May 3, 2012

(54) REAGENT FOR MEASUREMENT OF CHOLINESTERASE ACTIVITY

(75) Inventors: Tatsuya Kikuchi, Chiba (JP); Toshiaki Irie, Chiba (JP); Kiyoshi Fukushi, Chiba (JP); Toshimitsu Okamura, Chiba (JP)

(73) Assignee: NATIONAL INSTITUTE OF RADIOLOGICAL SCIENCES, Chiba-shi, Chiba (JP)

(21) Appl. No.: 13/255,051

(22) PCT Filed: Mar. 3, 2009
(86) PCT No.: PCT/JP2009/053928

§ 371 Date: Jan. 12, 2012

(51) Int. Cl. C12Q 1/46; C07D 211/20

(52) U.S. Cl. 435/20; 546/248

(57) ABSTRACT

An N-alkylpiperidine methanethiol ester derivative represented by any one of the general formulae (1) to (4) or a salt thereof has been discovered. The N-alkylpiperidine methanethiol ester derivative or the salt thereof has specificity for acetylcholinesterase or butyrylcholinesterase and is useful as a reagent for measurement of cholinesterase activity by the Ellman method.

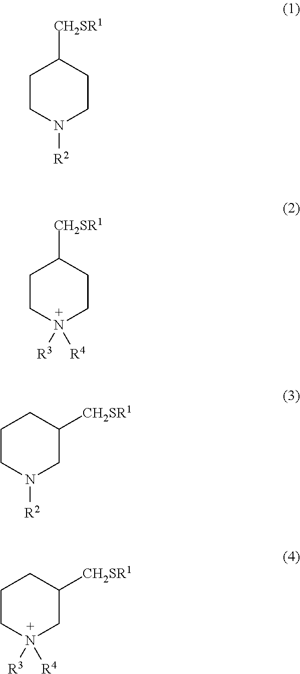

### TECHNICAL FIELD

The present invention relates to a novel piperidine methanethiol ester derivative and a salt thereof, as well as a method for producing the same. Moreover, the present invention relates to use of the compound as a reagent for measurement of cholinesterase activity.

### BACKGROUND ART

Cholinesterase is an enzyme which degrades a choline ester into choline and a carboxylic acid. There have been known two kinds of cholinesterase, namely acetylcholinesterase and butyrylcholinesterase.

Acetylcholinesterase is an enzyme which degrades acetylcholine. Measurement of acetylcholinesterase activity has been widely used in screening for exposure to agricultural chemicals, pesticides, chemical warfare agents, and the like, development of therapeutic drugs against Alzheimer's disease where the activity of the enzyme is lowered, and evaluation on nervous system disorders, and has been increasingly important recently.

Butyrylcholinesterase is an enzyme which degrades choline esters including acetylcholine. Since butyrylcholinesterase is biosynthesized in the liver, and released to the blood, the measurement of the enzyme in the blood makes it possible to obtain useful indicators for diagnosis, treatment, and the like of liver function, physical condition in the use of an anticholinesterase agent, organic phosphorus poisoning, nephrotic syndrome, hyperthyroidism, and the like. Therefore, the measurement of butyrylcholinesterase activity is an important measurement parameter in the field of clinical diagnosis.

For the measurement of cholinesterase activity, the Ellman method (see Non-Patent Document 1) has been widely used in which a thiocholine derivative is used, and the amount of a produced hydrolytic metabolite is measured on the basis of the absorbance of the yellow color by 2-nitro-5-mercaptobenzoic acid liberated through a reaction of the thiocholine derivative with 5,5′-dithio-bis(2-nitrobenzoic acid).

In addition to this method, there are a method in which a radioactively labeled choline ester derivative is used, and the amount of a produced radioactive hydrolytic metabolite is measured by liquid scintillation; and a method in which the amount of a carboxylic acid generated through hydrolysis of a non-radioactively labeled choline ester is measured by use of phenolphthalein, which is a pH indicator, or by ion chromatography, or the like (for example, see Patent Document 1).

However, any of conventionally known substrates used in these measurements of acetylcholinesterase activity has poor specificity, which necessitates the addition of a reagent for inhibiting butyrylcholinesterase. This results in a complicated and unstable measurement.

To overcome the problem of the specificity, there is a measurement method using 1-methyl-4-piperidinol acetyl ester labeled with carbon 14 or fluorine 18, or the like (for example, see Patent Document 2, and Non-Patent Documents 2 to 5). However, the use of an radioisotope has problems in terms of convenience, safety, and economy, such as a problem that the operations of measurement and the handling of samples are complicated, and a problem the that, in order to prevent unforeseen risks, the measurement method is allowed to be implemented only in facilities licensed to handle radiation with high equipment costs, for example.
Patent Document 1: Japanese Patent Application Publication No. Hei 10-253611
Patent Document 2: Japanese Patent Application Publication No. 2005-22996
Non-Patent Document 1: Biochemical Pharmacology 7, 88-95(1961)
Non-Patent Document 2: Nucl. Med. Biol. Vol. 21, NO. 6, pp. 801-808 (1994)
Non-Patent Document 3: J. Labelled Cpd. Radiopharm, 44, 31-41 (2001)
Non-Patent Document 4: Bioorganic & Medicinal Chemistry Letters 14, 1927-1930 (2004)
Non-Patent Document 5: J. Nucl. Med. 37: 649-655 (1996)

### SUMMARY OF INVENTION

### Technical Problems

An object of the present invention is to provide a novel compound useful as a reagent which enables measurement specific for activities of cholinesterases including acetylcholinesterase and butyrylcholinesterase, particularly for acetylcholinesterase activity, and to provide a method for producing the compound.

Another object of the present invention is to provide a reagent, which is excellent in terms of convenience, safety, and economy, for measurement of activities of cholinesterases including acetylcholinesterase and butyrylcholinesterase without the use of a radioisotope, and to provide a method for measurement of activities of cholinesterases using the reagent.

### Means to Solve Problems

To achieve the above-described objects, the present inventors have conducted study on a substrate used for measurement of cholinesterase activity without the use of a radioactive substance. As a result, it has been found that it is possible to produce primary thio esters, N-alkylpiperidine methanethiol ester derivatives represented by the following general formulae (1) to (4) which cannot be produced by conventional producing methods, as well as to produce salts thereof, and that the compounds are substrates having specificity for acetylcholinesterase or butyrylcholinesterase, i.e., are hydrolyzed specifically with one of the enzymes.

In addition, it has been found that, in particular, the compounds of the following general formulae (1) to (4), where R¹ is an acetyl group, and R² to R⁴ are each a methyl group, exhibits such characteristics that the compounds are hydrolyzed with acetylcholinesterase, but are hardly hydrolyzed with butyrylcholinesterase (i.e., have specificity for acetylcholinesterase).

Moreover, it has been found that the compounds of the following general formulae (1) to (4) are highly stabile in an aqueous solution, and hence are usable as substrates for the Ellman method.

As described above, each of the compounds of the present invention is stable in an aqueous solution, is hydrolyzed specifically with acetylcholinesterase or butyrylcholinesterase, and has a primary thio ester in the molecule. Hence, the compound is particularly useful as a reagent for the Ellman method which enables the measurement of cholinesterase activity by absorbance analysis.

The piperidine compounds having the thio ester structure of the present invention are compounds for which no effective producing method has been reported so far, and which have been found by the present inventors for the first time.

The present invention provides the following.

1. An N-alkylpiperidine methanethiol ester derivative represented by any one of the following general formulae (1) to (4), or a salt thereof:

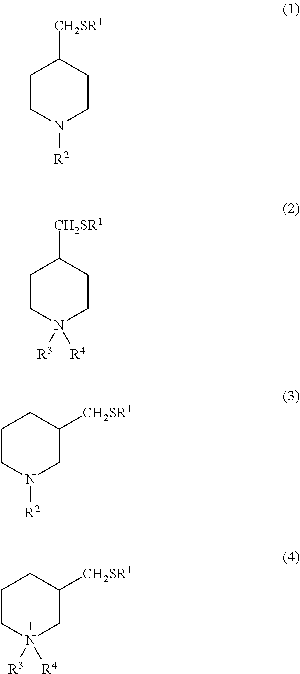

wherein R⁴ represents an acyl group represented by COR¹′ (where R¹′ represents an alkyl group having 1 to 4 carbon atoms), and R², R³, and R⁴ each represents hydrogen or an alkyl group having 1 or 2 carbon atoms.
2. The piperidine methanethiol ester derivative or the salt thereof according to the above-described 2, wherein

R¹ is an acetyl group, a propionyl group, a butyryl group, or a valeryl group.

3. The piperidine methanethiol ester derivative or the salt thereof according to above-described 1 or 2, wherein

R², R³, and R⁴ are each a methyl group.

4. The piperidine methanethiol ester derivative or the salt thereof according to the above-described 3, wherein

R¹ is an acetyl group, and

R², R³, and R⁴ are each a methyl group.

5. A reagent for measurement of acetylcholinesterase or butyrylcholinesterase activity, comprising the piperidine methanethiol ester derivative or the salt thereof according to any one of the above-described 1 to 4.
6. A reagent for measurement of acetylcholinesterase activity, comprising the piperidine methanethiol ester derivative or the salt thereof according to the above-described 4.
7. Use of the piperidine methanethiol ester derivative or the salt thereof according to any one of the above-described 1 to 4 as a reagent for measurement of acetylcholinesterase or butyrylcholinesterase activity.
8. Use of the piperidine methanethiol ester derivative or the salt thereof according to the above-described 4 as a reagent for measurement of acetylcholinesterase activity.
9. A method for producing the compound of formula (1) or (3) according to any one of the above-described 1 to 4, comprising:

step (a) of obtaining a 1-alkyl-4-piperidine methanethiol or a 1-alkyl-3-piperidine methanethiol from a 1-alkyl-4-piperidine methanol or a 1-alkyl-3-piperidine methanol; and

step (b) of acylating the 1-alkyl-4-piperidine methanethiol or the 1-alkyl-3-piperidine methanethiol in the presence of a base by use of an acylating agent, to thereby produce a 1-alkylpiperidin-4-ylmethyl acyl sulfide (1) or a 1-alkylpiperidin-3-ylmethyl acyl sulfide (3).

10. A method for producing the compound of formula (2) or (4) according to any one of the above-described 1 to 4, comprising:

step (a) of obtaining a 1-alkyl-4-piperidine methanethiol or a 1-alkyl-3-piperidine methanethiol from a 1-alkyl-4-piperidine methanol or a 1-alkyl-3-piperidine methanol;

step (b) of acylating the 1-alkyl-4-piperidine methanethiol or the 1-alkyl-3-piperidine methanethiol in the presence of a base by use of an acylating agent, to thereby produce a 1-alkylpiperidin-4-ylmethyl acyl sulfide (1) or a 1-alkylpiperidin-3-ylmethyl acyl sulfide (3); and

step (c) of heating the 1-alkylpiperidin-4-ylmethyl acyl sulfide (1) or the 1-alkylpiperidin-3-ylmethyl acyl sulfide (3) in the presence of an alkylating agent in an solvent, to thereby obtain a 1,1-dialkylpiperidin-4-ylmethyl acyl sulfide (2) or a 1,1-dialkylpiperidin-3-ylmethyl acyl sulfide (4).

### Advantageous Effects of Invention

The compound of the present invention, in particular, the compound of any one of the following formulae (1) to (4), where R¹ is an acetyl group, has high specificity for acetylcholinesterase. Moreover, it is possible to impart specificity for butyrylcholinesterase by increasing the number of carbon atoms in the acyl group of R¹. Hence, the compound is useful also as a reagent for measurement specific for activities of both the enzymes.

The compound of the present invention gives a thiol group upon hydrolysis. Hence, the compound can be advantageously used especially for measurement of activities of cholinesterases by the Ellman method (Biochemical Pharmacology 7, 88-95 (1961)). Accordingly, it is not necessary to use a radioactive substance, which complicates the operations of measurement and the handling of samples, and, in order to prevent unforeseen risks, and which can be used only in facilities licensed to handle radiation with high equipment costs. Hence, the compound of the present invention is excellent in terms of convenience, safety, and economy.

### DESCRIPTION OF EMBODIMENTS

### <Compound>

The compound of the present invention is an N-alkylpiperidine methanethiol ester derivative represented by any one of the following general formulae (1) to (4), or a salt thereof:

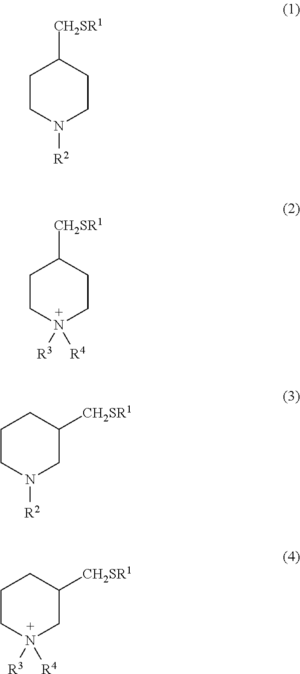

wherein R¹ represents an acyl group represented by COR¹′ (where R¹′ represents an alkyl group having 1 to 4 carbon atoms), and R², R³, and R⁴ each represents hydrogen or an alkyl group having 1 or 2 carbon atoms.

R² to R⁴ each may be an alkyl group having a radioactive element such as ¹⁴C. The present invention has an advantage that cholinesterase activity can be measured without using a radioactive element, but, as a matter of course, a person skilled in the art understands that the cholinesterase activity can be measured also when a radioactively labeled compound is used.

Regarding the stereochemistry of the carbon atom in the 3-position on the piperidine ring of the compound of each of the formulae (3) and (4), any one of S, R, and racemic isomers may be employed.

Examples of the acyl group of R¹ include an acetyl group, a propionyl group, a butyryl group, and a valeryl group, and an acetyl group is particularly preferable. Meanwhile, R², R³, and R⁴ are preferably each a methyl group.

The compound of the formula (1) or (2), where R¹ is an acetyl group, and R² to R⁴ are each a methyl group, has extremely high specificity for acetylcholinesterase, and is hence particularly preferable.

By increasing the number of carbon atoms in the acyl group of R¹, specificity for butyrylcholinesterase can also be imparted. In particular, cases where R¹ is a butyryl group or a valeryl group are preferable, because a high specificity for butyrylcholinesterase is obtained.

Examples of the salt of each of the compounds of formula (1) and (3) of the present invention include pharmacologically acceptable salts such as hydrochloric acid salts, sulfuric acid salts, and acetic acid salts. Examples of the salt of each of the compounds of formula (2) and (4) of the present invention include pharmacologically acceptable salts such as chloride salts, bromide salts, and iodide salts.

### <Method for Measuring Acetylcholinesterase or Butyrylcholinesterase Activity>

The compound of the present invention can be used as a reagent for a method for measuring acetylcholinesterase or butyrylcholinesterase activity.

An example of the method for measuring acetylcholinesterase or butyrylcholinesterase activity is a measurement method by the Ellman method (Biochemical Pharmacology 7, 88-95 (1961)) characterized by the use of a solution of 5,5′-dithio-bis(2-nitrobenzoic acid).

The Ellman method can be described, in brief, as a method comprising: a step of mixing a test solution containing a cholinesterase, the compound of the present invention, and a solution of 5,5′-dithio-bis(2-nitrobenzoic acid); and a step of quantifying color development by 5-thio-2-nitrobenzoic acid derived from the solution of 5,5′-dithio-bis(2-nitrobenzoic acid).

Further details are as follows. When the test solution containing a cholinesterase, the compound of the present invention, and the solution of 5,5′-dithio-bis(2-nitrobenzoic acid) are mixed with each other, the compound of the present invention is hydrolyzed with the cholinesterase to give a thiol compound. When the thiol compound and 5,5′-dithio-bis(2-nitrobenzoic acid) react with each other, the residue, 5-thio-2-nitrobenzoic acid, dissolves in the solution, and develops the color. By quantifying the color development, the hydrolysis rate or the hydrolysis ratio of the compound of the present invention is quantified. Thus, the cholinesterase activity can be measured.

In general, 5,5′-dithio-bis(2-nitrobenzoic acid) is used, for example, after being dissolved in a solvent such as a phosphate buffer at a concentration of about 0.2 to 1 mM, but the use thereof is not limited thereto.

Meanwhile, the compound of the present invention is generally used after being dissolved in a solvent such as a phosphate buffer at a concentration of about 0.5 to 1 mM, but the use thereof is not limited thereto.

The color development of 5-thio-2-nitrobenzoic acid can be quantified by a conventionally known method. For example, the quantification can be carried out by measuring the absorbance (mAbs) at a wavelength of 412 nm or 436 nm (Clinica Chimica Acta 288, 73-90 (1999)).

Note that a modified method of the Ellman method is also reported, and the measurement by the modified method can be carried out by using a solution of 2,2′- or 4,4′-dithiodipyridine (Rinsho Byori: The Official Journal of Japanese Society of Laboratory Medicine, 350-354 (1971)) instead of 5,5′-dithio-bis(2-nitrobenzoic acid).

Besides the use of the compound of the present invention for the above-described measurement methods, the compound of the present invention can be used in the same manner as in the case of acetyl thiocholine, which has been conventionally used as a reagent for measurement of acetylcholinesterase activity or butyrylcholinesterase activity.

### <Method for Producing Compound of the Present Invention>

The thiol compound of the present invention is synthesized by the following production route for the first time.

Conventionally, a substituent is introduced into a piperidine methanol compound as follows. Specifically, the amino group on the piperidine ring is protected in advance; then, acylation or the like is conducted on the hydroxymethyl group in the 3- or the 4-position; thereafter, the protective group is deprotected; and an alkyl group is introduced onto the amino group (for example, Bioorganic & Medicinal Chemistry Letters 14, 1927-1930 (2004)). However, it is not possible to introduce a substituent into a piperidine methanethiol compound by such a method. This is because the introduction method requires using a piperidine methanethiol compound as a starting material for the synthesis, but no method for obtaining a piperidine methanethiol compound has conventionally been known. The present inventors have found that the thiol compound of the present invention can be produced by applying the piperidine methanethiol synthesis method reported by R. Cao, Jr et al., (J. Am. Chem. Soc., 129, 6927-6930, 2007) through the following steps via a 1-alkylpiperidine methanethiol.

In the method for producing the compound of formula (1) or (3), the compound of formula (1) or (3) can be produced by a method comprising the following steps: step (a) of obtaining a 1-alkyl-4-piperidine methanethiol or a 1-alkyl-3-piperidine methanethiol from a 1-alkyl-4-piperidine methanol or a 1-alkyl-3-piperidine methanol; and step (b) of acylating the 1-alkyl-4-piperidine methanethiol or the 1-alkyl-3-piperidine methanethiol in the presence of a base by use of an acylating agent, to thereby produce a 1-alkylpiperidin-4-ylmethyl acyl sulfide (1) or a 1-alkylpiperidin-3-ylmethyl acyl sulfide (3).

In the method for producing the compound of formula (2) or (4), the compound of formula (2) or (4) can be produced by a method comprising the following steps: step (a) of obtaining a 1-alkyl-4-piperidine methanethiol or a 1-alkyl-3-piperidine methanethiol from a 1-alkyl-4-piperidine methanol or a 1-alkyl-3-piperidine methanol; step (b) of acylating the 1-alkyl-4-piperidine methanethiol or the 1-alkyl-3-piperidine methanethiol in the presence of a base by use of an acylating agent, to thereby produce a 1-alkylpiperidin-4-ylmethyl acyl sulfide (1) or a 1-alkylpiperidin-3-ylmethyl acyl sulfide (3); and step (c) of heating the 1-alkylpiperidin-4-ylmethyl acyl sulfide (1) or the 1-alkylpiperidin-3-ylmethyl acyl sulfide (3) in the presence of an alkylating agent in a solvent, to thereby obtain a 1,1-dialkylpiperidin-4-ylmethyl acyl sulfide (2) or a 1,1-dialkylpiperidin-3-ylmethyl acyl sulfide (4).

Each of the steps is described in detail.

Step (a)

As a starting substance, a 1-alkyl-4-piperidine methanol or a 1-alkyl-3-piperidine methanol is used, which is commercially available, or which can be obtained by causing 4-piperidine methanol or 3-piperidine methanol to react with an alkyl halide in the presence of a base in the same manner as in step (c). The compound is dissolved in an organic solvent such as acetonitrile or diisopropyl ether. To this solution, 1 to 1.5 molar equivalents of sodium sulfide is added, followed by stirring at room temperature to 70° C. for 1 to 12 hours. Thereafter, phosphoric acid (50 to 85%) is added dropwise, until the solution turns yellow. Eight to twenty-four hours later, the solvent is removed under vacuum. A buffer solution of pH 6.8 to 7.4 is added to the obtained residue. Extraction is conducted with a solvent such as dichloromethane, and further the solvent is removed under vacuum. Thus, the 1-alkyl-4-piperidine methanethiol or the 1-alkyl-3-piperidine methanethiol is obtained. The obtained 1-alkyl-4-piperidine methanethiol or 1-alkyl-3-piperidine methanethiol may further be purified, or may be used as it is in the next step (b) without purification.

Step (b)

The 1-alkyl-4-piperidine methanethiol or the 1-alkyl-3-piperidine methanethiol is allowed to react with an acylating agent such as acetic anhydride or acetyl chloride in the presence of a base such as pyridine or triethylamine in a solvent at a temperature of from room temperature to about 80° C. for about 1 to 6 hours. Examples of the solvent used at this time include dichloromethane and the like. Next, desalination is conducted by use of an anhydrous base such as ammonia-containing chloroform. Thus, the 1-alkylpiperidin-4-ylmethyl acyl sulfide (1) or the 1-alkylpiperidin-3-ylmethyl acyl sulfide (3) is obtained.

Step (c)

The 1-alkylpiperidin-4-ylmethyl acyl sulfide (1) or the 1-alkylpiperidin-3-ylmethyl acyl sulfide (3) is heated together with an alkylating agent such as methyl halide, for example, in a solvent such as diisopropyl ether or dimethylformamide, for example, at room temperature to 130° C. (for example, 40° C.) for 6 to 24 hours. Thus, the 1,1-dialkylpiperidin-4-ylmethyl acyl sulfide (2) or the 1,1-dialkylpiperidin-4-ylmethyl acyl sulfide (4) is obtained.

By the production method described above, for example, 1,1-dimethylpiperidin-4-ylmethyl acyl sulfide (2a) was successfully obtained.

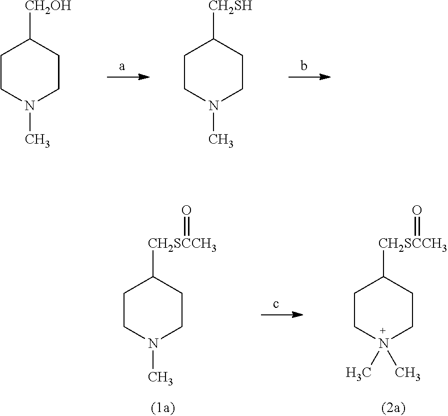

A synthetic route to the compound of formula (2a) is briefly described.

### Step (a)

From commercially available 1-methyl-4-piperidine methanol, 1-methyl-4-piperidine methanethiol can be obtained by the method known from the publication (R. Cao, Jr, et al., J. Am. Chem. Soc., 129, 6927-6930, 2007).

### Step (b)

In the presence of pyridine, triethylamine, or the like, 1-methyl-4-piperidine methanethiol is allowed to react with acetic anhydride or acetyl chloride at room temperature for about 1 to 2 hours, followed by desalination by use of ammonia-containing chloroform. Thus, 1-methylpiperidin-4-ylmethyl acetyl sulfide (1a) can be obtained.

### Step (c)

For example, in a solvent such as diisopropyl ether, 1-methylpiperidin-4-ylmethyl acetyl sulfide (1a) is heated with methyl iodide or the like, for example, at 40° C. for 12 to 14 hours. Thus, 1,1-dimethylpiperidin-4-ylmethyl acetyl sulfide (2a) can be obtained.

### EXAMPLES

Next, the present invention is described more specifically by showing Example 1

Into 150 ml of acetonitrile, 1 g (7.7 mmol) of commercially available 1-methyl-4-piperidine methanol was dissolved, and 800 mg (10.3 mmol) of sodium sulfide was added thereto, followed by stirring at 40° C. for 2 hours. Then, phosphoric acid was added dropwise until the solution turned yellow. Twelve hours later, the solvent was removed under vacuum, and a phosphate buffer (0.1 M) of pH 7.4 was added to the obtained residue, followed by extraction with dichloromethane four times. The dichloromethane solution was dried over anhydrous magnesium sulfate, and concentrated by evaporation under vacuum. Then, 400 mg (5.1 mmol) of acetyl chloride was added thereto, followed by stirring for 1 hour at room temperature. The solvent in the reaction solution was removed under vacuum, and the obtained residue was dissolved in 5 ml of chloroform. To this solution being cooled on ice, 5 ml of a saturated ammonia/chloroform solution was added. Insolubles were removed by filtration, and the solvent was removed under vacuum. The obtained residue was purified by column chromatography (NH silica gel, Eluent hexane:ethyl acetate=4:1). Thus, 143 mg of 1-methylpiperidin-4-ylmethyl acetyl sulfide (1a) was obtained as a brown oily substance (Yield: 10%).

FAB-MS (m/z): (M⁺+1) calcd for C₉H₁₇NOS, 188; found, 188

¹H NMR (300 MHz, CDCl₃) δ (ppm): 1.21-1.44 (3H, m, CH, CH×2), 1.71 (2H, d, CH₂, J=15.0 Hz), 1.82-1.90 (2H, m, CH×2), 2.21 (3H, s, CH₃), 2.28 (3H, s, CH₃), 2.76-2.81 (4H, m, CH×4);

¹³C NMR (300 MHz, CDCl₃) δ (ppm): 30.58, 31.45, 35.03, 35.45, 46.17, 55.41, 195.70.

### Example 2

Into 15 ml of diisopropyl ether, 250 mg (1.3 mmol) of 1-methylpiperidin-4-ylmethyl acetyl sulfide (1a) was dissolved, and 500 mg (3.5 mmol) of methyl iodide was added to the solution, followed by a reaction at 40° C. for 12 hours. The solvent was removed under vacuum. The obtained residue was recrystallized from ethanol, and vacuum filtration was conducted. Thus, 76.2 mg of the iodide salt of 1,1-dimethylpiperidin-4-ylmethyl acetyl sulfide (2a) was obtained as light brown crystals (Yield: 18%).

FAB-MS (m/z): (Mt) calcd for C₁₀H₂₀NOS, 202; found, 202

¹H NMR (300 MHz, CD₃OD) δ (ppm): 1.67-1.97 (5H, m, CH, CH×4), 2.34 (3H, s, CH₃), 2.96 (2H, d, CH₂, J=6.0 Hz), 3.12 (3H, s, CH₃), 3.18 (3H, s, CH₃), 3.36-3.53 (4H, m, CH×4);

¹³C NMR (300 MHz, CD₃OD) δ (ppm): 26.52, 30.50, 34.18, 34.76, 56.38, 63.26, 196.78.

### Example 3

### Enzymatic Reactions Using Compounds (1a) and (2a) and Specificities Thereof

To investigate the hydrolysis rate and specificity of each of (1a) and (2a), the hydrolysis rate in a solution of each of purified human acetylcholinesterase and purified human butyrylcholinesterase was measured by the Ellman method, and was compared with those of acetyl thiocholine and acetyl-δ methyl-thiocholine. To 3 ml of a solution of each enzyme whose concentration was adjusted with a phosphate buffer (0.1 M, pH 7.4) containing 0.1% of Tween 20, 0.1 ml of a solution (5 mM) of 5,5′-dithio-bis(2-nitrobenzoic acid) was added. To these mixtures, the substrates were respectively added in an amount of 0.02 ml (75 mM), and the absorbance (mAbs) at a wavelength of 412 nm was measured.

Table 1 shows the measurement results.

Table 1 shows the hydrolysis rates of acetyl thiocholine, acetyl-δ methyl-thiocholine, and the compounds (1a) and (2a) with acetylcholinesterase and butyrylcholinesterase, as well as spontaneous hydrolysis rates thereof. The values in the Table represent the changes in absorbance per minute measured three times in the form of average value±standard deviation.

TABLE 1

| | Acetylcho- | Butyrylcho- | spontaneous |
|---|---|---|---|
| | linesterase | linesterase | hydrolysis |
| Acetyl thiocholine | 37 ± 0.37 | 18 ± 0.11 | 0.21 ± 0.021 |
| Acetyl-β | 29 ± 0.24 | 7.1 ± 0.22 | 0.48 ± 0.020 |
| methyl-thiocholine | | | |
| Compound (1a) | 3.7 ± 0.098 | 0.47 ± 0.026 | 0.47 ± 0.0096 |
| Compound (2a) | 3.1 ± 0.15 | 0.15 ± 0.0077 | 0.13 ± 0.027 |

As shown in Table 1, the spontaneous hydrolysis rate (ΔmAbs/minute) of each of the substrates in the buffer solution was low.

Moreover, the hydrolysis rate of each of acetyl thiocholine and acetyl-δ methyl-thiocholine with butyrylcholinesterase was significantly higher than that of the spontaneous hydrolysis in the buffer solution. In contrast, the hydrolysis rate of each of the compounds (1a) and (2a) with butyrylcholinesterase did not show any significant difference with respect to the spontaneous hydrolysis rate. This indicates that butyrylcholinesterase causes substantially no hydrolysis of the compounds (1a) and (2a).

In addition, it has been shown that the hydrolysis rate of each of the compounds (1a) and (2a) with acetylcholinesterase was lower than the hydrolysis rates of acetyl thiocholine and acetyl-δ methyl-thiocholine, but the compounds (1a) and (2a) were good substrates for acetylcholinesterase. As described above, it was shown that the compounds (1a) and (2a) were extremely highly specific for acetylcholinesterase.

### Example 4

### Time Dependence of Enzymatic Reactions of (1a) and (2a)

To demonstrate that the hydrolysis rate of each of (1a) and (2a) is consistent with the zero-order reaction, reactions were conducted in the same manner as in the above-described example, and the absorbance at wavelength of 412 nm was measured in a time-dependent manner. As a result, the absorbance changed by approximately 0.4 Abs in 120 minutes, and the change with time showed a good linearity. Hence, it was found that the hydrolysis rate of each substrate was consistent with the zero-order reaction at the substrate concentration (FIG. 1). The values represent the absorbance (Abs) measured three times in the form of average value±standard deviation. In FIG. 1, “o” shows the hydrolysis rate of the compound (1a), and “□” shows the hydrolysis rate of the compound (2a).

### BRIEF DESCRIPTION OF DRAWING(S)

FIG. 1 shows time dependence of the hydrolysis rates of the compounds (1a) and (2a) with acetylcholinesterase.

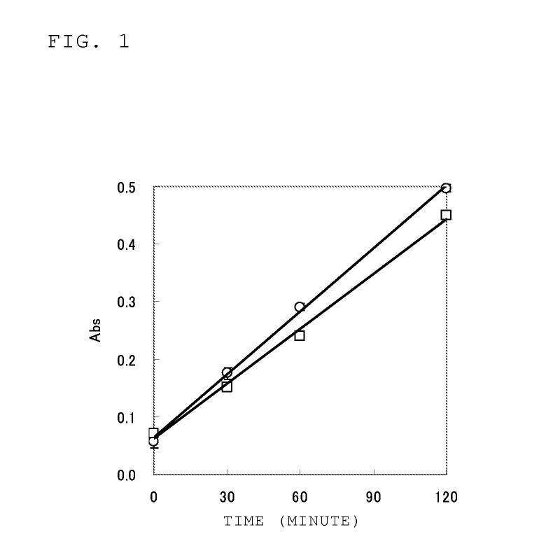

1. An N-alkylpiperidine methanethiol ester derivative represented by any one of the following general formulae (1) to (4), or a salt thereof: 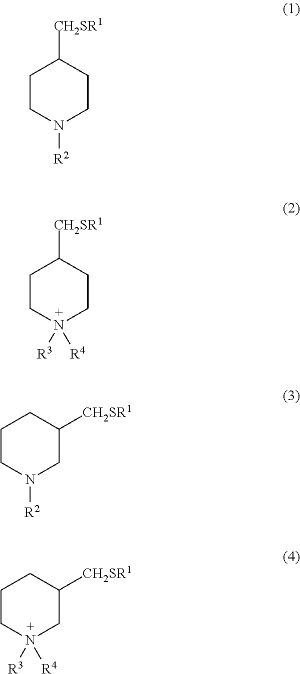
wherein R¹ represents an acyl group represented by COR¹′ (where R¹′ represents an alkyl group having 1 to 4 carbon atoms), and R², R³, and R⁴ each represents hydrogen or an alkyl group having 1 or 2 carbon atoms.

2. The N-alkylpiperidine methanethiol ester derivative or the salt thereof according to claim 1, wherein
R¹ is an acetyl group, a propionyl group, a butyryl group, or a valeryl group.

3. The N-alkylpiperidine methanethiol ester derivative or the salt thereof according to claim 1, wherein
R², R³ and R⁴ are each a methyl group.

4. The N-alkylpiperidine methanethiol ester derivative or the salt thereof according to claim 1, wherein
R¹ is an acetyl group, and
R², R³ and R⁴ are each a methyl group.

5-10. (canceled)

11. A method for measuring cholinesterase activity in a test solution, comprising steps of:
(i) mixing the test solution containing a cholinesterase, a compound as set forth in claim 1 and a solution of 5,5′-dithio-bis(2-nitrobenzoic acid);
(ii) conducting an absorbance analysis of the test solution obtained in step (i) to determine the hydrolysis ratio of the compound as set forth in claim 1 to determine cholinesterase activity in the test solution.

12. The method of claim 11, wherein the step of (ii) conducting an abosrbance analysis contains a step of (ii-1) quantifying the absorbance (mAbs) at a wavelength of 412 nm of the solution obtained in step (i).

13. The method of claim 11, wherein the compound to be mixed in step (i) is a compound wherein R¹ is an acetyl group, a propionyl group, a butyryl group, or a valeryl group.

14. The method of claim 11, wherein the compound to be mixed in step (i) is a compound wherein R², R³ and R⁴ are each a methyl group.

15. A method for measuring acetylcholinesterase activity in a test solution, comprising steps of:
(i) mixing the test solution containing an acetylcholinesterase, a compound as set forth in claim 4 and a solution of 5,5′-dithio-bis(2-nitrobenzoic acid);
(ii) conducting an absorbance analysis of the test solution obtained in step (i) to determine the hydrolysis ratio of the compound as set forth in claim 4 to determine acetylcholinesterase activity in the test solution.
